(19) United States
(12) Patent Application Publication
(10) Pub. No.: US 2008/0248957 A1
(43) Pub. Date: Oct. 9, 2008

(54) Pharmaceutical Composition

(75) Inventors: Ernest Schay, Edenvale (ZA); Walter Focke, Pretoria (ZA); Lushane Walbrugh, Gemiston (ZA)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: BAYER HEALTHCARE AG, LEVERKUSEN (DE)

(21) Appl. No.: 11/574,955

(22) PCT Filed: Aug. 27, 2005
(86) PCT No.: PCT/EP2005/009266

§ 371 Date: Feb. 29, 2008

(30) Foreign Application Priority Data
Sep. 9, 2004 (ZA) 2004/7207

(51) Int. Cl. A01N 25/00; A61K 47/16; A01N 35/10; A01P 7/02; A01P 13/00

(52) U.S. Cl. 504/358; 514/788; 514/636

(57) ABSTRACT

A pharmaceutical composition is provided comprising an active pharmaceutical composition and a water-soluble eutectic composition, the resultant mixture being formed into a solid, water-soluble dosage form.

### FIELD OF THE INVENTION

This invention relates to a pharmaceutical composition and, more particularly, to a pharmaceutical composition in the form of a water-soluble solid dosage form.

### BACKGROUND TO THE INVENTION

In this specification the term “pharmaceutical” shall have its widest meaning and include compounds used in the treatment of humans, animals and plants. This term includes pesticides and in this specification the term “pesticide” is intended to encompass within its scope herbicides and compositions for eradicating or controlling animal pests.

Liquids in general, and pesticides in particular, are usually sold as a liquid concentrate in metal or plastic containers from which a desired quantity of the product is decanted before use.

Pesticides, by their nature, are hazardous chemicals and disposal of empty pesticide containers is a problem as the containers usually contain a pesticide residue which can leach into the environment with disastrous consequences. To an extent, similar problems can be experienced with full or partially full containers, particularly metal containers or plastic containers that have stripped cap screws. Also, containers frequently fall and break during transport.

Another problem with the containers is the use, particularly in developing countries where amenities such as running water are lacking, of empty containers to store water for domestic use. The dangers of such a practise need no elaboration.

One attempt to address the above problem involves supplying the active pesticide ingredient in powder form in plastic bags. A desired quantity of the active is measured out and mixed with water shortly before using. To a large extent the plastic bags, while requiring less storage space than metal or plastic containers, suffer from the same disadvantages as the containers particularly when they are stored in damp environments and also when measuring the correct quantity of the powder.

### OBJECT OF THE INVENTION

It is an object of this invention to provide a pharmaceutical composition and, more particularly, to provide a pharmaceutical composition in the form of a water-soluble solid dosage form which at least partly alleviates the above-mentioned disadvantages.

### SUMMARY OF THE INVENTION

In accordance with this invention there is provided a pharmaceutical composition comprising an active pharmaceutical composition and a water-soluble eutectic composition, the resultant mixture being formed into a solid, water-soluble dosage form.

There is further provided for the melting point of the eutectic composition to be lower than the melting point of the pharmaceutical composition.

In accordance with one aspect of the invention there is provided for the water-soluble eutectic composition to be a urea/1,3-dimethyl urea mixture, preferably in a 40%:60% m/m urea:dimethyl urea ratio.

There is further provided for the pesticide to be a herbicide. Alternatively there is provided for the pesticide to be suitable for controlling or eradicating mammalian pests, preferably ectoparasites and further preferably an acaricide.

There is also provided for the acaricide to be a pesticide belonging to the amidine group of pesticides, preferably AMITRAZ.

There is further provided for the pharmaceutical composition to include at least one suitable dispersant, and/or at least one disintegrant, and/or at least one surfactant, and/or at least one filler; for the dispersant to be a sodium salt of the condensation product of naphthalene sulphuric acid, preferably WETTOL D2; for the disintegrant to be selected from sodium starch glycolate and calcium carbonate; and for the surfactant to be a non-ionic surfactant belonging to the nonylphenol ethoxylate group, preferably ARKOPAL N090.

Still further according to this aspect of the invention there is provided for the composition to include AMITRAZ, calcium carbonate, ARKOPAL N090, WETTOL D2, urea, 1,3-dimethyl urea, sodium starch glycolate and polyethylene glycol; and for the constituents to be present in the following ranges (% m/m):

| | | |
|---|---|---|
| AMITRAZ | 30 to 40% | m/m |
| Calcium carbonate | 8 to 11% | m/m |
| ARKOPAL N090 | 2 to 3% | m/m |
| WETTOL D2 | 1 to 2% | m/m |
| Urea | 10 to 20% | m/m |
| 1,3-dimethyl urea | 15 to 30% | m/m |
| Sodium starch glycolate | 5 to 15% | m/m |
| Polyethylene glycol | 0 to 15% | m/m |

The invention also provides a method of manufacturing a pharmaceutical composition in solid dosage form which includes melting a water-soluble eutectic composition, adding an active pharmaceutical composition and casting the resultant mixture into moulds.

### BRIEF DESCRIPTION OF ONE EMBODIMENT OF THE INVENTION

The invention will be described below by way of example only and with reference to the accompanying examples which are for acciricidal compositions.

### DETAILED DESCRIPTION OF EMBODIMENTS OF THE INVENTION

A pharmaceutical composition according to the invention is formed by mixing and heating urea and 1,3-dimethyl urea mixture having a 40% to 60% m/m ratio to 60° C., adding the acaricide, AMITRAZ milled together with calcium carbonate, and desired dispersants, surfactants and disintegrants to form a suspension. The resultant liquid is cast into solid dosage form shaped moulds and allowed to cool and solidify to produce a solid dosage form that is soluble in water. Each solid dosage form so produced is individually sealed with a plastics or foil material film.

Three different formulations were tested. The formulations were:

| | FORMULATION | | |
|---|---|---|---|
| | 1 | 2 | 3 |
| CONSTITUENT | % CONCENTRATION | | |
| AMITRAZ | 34.0 | 45.0 | 45.0 |
| CaCO₃ | 9.0 | 11.0 | 12.0 |
| ARKOPAL N090 | 2.0 | 3.0 | 3.0 |
| WETTOL | 1.1 | 1.1 | 1.1 |
| Urea | 11.0 | 15.0 | 15.0 |
| 1,3-dimethyl urea | 17.0 | 17.0 | 17.0 |
| Sodium starch glycolate | 25.0 | 10.0 | — |
| Polyethylene glycol | — | — | 10.0 |

The concentration of AMITRAZ was standard and based on an effective concentration to control or eradicate acarinids when the solid dosage form produced was dissolved in 10 litres of water. In each case, when added to 10 litres of water, the solid dosage form dissolved in approximately 5 minutes.

It is envisaged that the solid dosage forms can be used as a viable alternative to liquid and powder concentrates thus, at least partly, alleviating some of the disadvantages associated with the use of the liquid and powder. It is envisaged that a number of solid dosage forms can be added to any suitable volume of water to make up enough pesticide for application to one animal, in which case one solid dosage form of the pesticide can be made up in a bucket and sponged or sprayed onto the animal, or to several animals, in which case several solid dosage forms of the pesticide can be dissolved in a dip tank or in a spray race reservoir. Alternatively, instead of using several smaller solid dosage forms, one large (2-5 kg) dosage form could be produced and packed as described above.

Using a water soluble eutectic as a carrier in the manner described has the further advantage that it is easy to vary solid dosage form sizes. The mould is simply changed to a suitable size whereas with normal tableting equipment a change in tablet size generally requires expensive changes to dies and presses.

Also, by varying the amount of active ingredient and excipients in a formulation it is possible to control the dissolution rate of the solid dosage form. For example, increasing the concentration of AMITRAZ causes a slowing of the rate of dissolution. This makes it possible to provide a sustained release formulation where required, thus increasing the dissolution time from minutes to hours or days.

It will be appreciated that a wide variety of pesticides and additives can be used in the above invention without departing from the scope thereof. In particular, the pesticide active ingredient can be an acaricide as described or the pesticide may target other ectoparasites. Alternatively the pesticide active ingredient may target a variety of endoparasites such as nematodes and cystodes to name but two. Where the target is an endoparasite it is envisaged that the solid dosage form can be dissolved in water in a reservoir for an oral dosing gun.

In addition to the above, the pesticide active ingredient may also be a herbicide and the solid dosage form dissolved in water in a suitable reservoir shortly before it is applied. Additionally, the active ingredient may also be a protein or disinfectant, or any other pharmacologically active material.

1. A pharmaceutical composition comprising an active pharmaceutical composition and a water-soluble eutectic composition, the resultant mixture being formed into a solid, water-soluble dosage form.

2. A pharmaceutical composition as claimed in claim 1 in which the melting point of the eutectic composition is lower than the melting point of the pharmaceutical composition.

3. A pharmaceutical composition as claimed in claim 1 or claim 2 in which the eutectic composition is a urea and 1,3-dimethyl urea mixture.

4. A pharmaceutical composition as claimed in claim 3 in which the urea:dimethyl urea ratio is 40% m/m:60% m/m.

5. A pharmaceutical composition as claimed in claim 3 or claim 4 in which the urea and 1,3-dimethyl urea mixture is present in the range 25 to 50 % m/m.

6. A pharmaceutical composition as claimed in any one of the preceding claims in which the active pharmaceutical composition is a pesticide.

7. A pharmaceutical composition as claimed in claim 6 in which the pesticide is a herbicide.

8. A pharmaceutical composition as claimed in claim 7 in which the pesticide is suitable for controlling or eradicating mammalian pests.

9. A pharmaceutical composition as claimed in claim 8 in which the pesticide is suitable for controlling or eradicating ectoparasites.

10. A pharmaceutical composition as claimed in claim 8 in which the pesticide is an acaricide.

11. A pharmaceutical composition as claimed in claim 10 in which the acaricide belongs to the amidine group of pesticides.

12. A pharmaceutical composition as claimed in claim 11 in which the pesticide is AMITRAZ.

13. A pharmaceutical composition as claimed in claim 12 in which the AMITRAZ is present in the range 30 to 40 % m/m.

14. A pharmaceutical composition as claimed in any one of the preceding claims which further includes any one or more of at least one dispersant, at least one disintegrant, at least one surfactant and at least one filler.

15. A pharmaceutical composition as claimed in claim 14 in which the dispersant is a sodium salt of the condensation product of naphalene sulphuric acid.

16. A pharmaceutical composition as claimed in claim 15 in which the dispersant is WETTOL D2.

17. A pharmaceutical composition as claimed in any one of claims 14 to 16 in which the dispersant is present in the range 1 to 2 % m/m.

18. A pharmaceutical composition as claimed in any one of claims 14 to 17 in which the surfactant is a non-ionic surfactant belonging to the nonylphenol ethoxylate group.

19. A pharmaceutical composition as claimed in claim 18 in which the surfactant is ARKOPAL N090.

20. A pharmaceutical composition as claimed in any one of claims 14 to 19 in which the dispersant is present in the range 2 to 3 % m/m.

21. A pharmaceutical composition as claimed in any one of claims 14 to 20 in which the disintegrant is sodium starch glycolate.

22. A pharmaceutical composition as claimed in claim 21 in which the sodium starch glycolate is present in the range 5 to 15 % m/m.

23. A pharmaceutical composition as claimed in any one of claims 14 to 22 which includes calcium carbonate present in the range 8 to 11 % m/m.

24. A method of manufacturing a pharmaceutical composition in solid dosage form which includes melting a water-soluble eutectic composition, adding an active pharmaceutical composition and casting the resultant mixture into moulds.

25. A method as claimed in claim 24 which further includes adding any one or more of at least one dispersant, at least one disintegrant, and at least one surfactant to the melted polymeric composition.

26. A method as claimed in claim 24 or claim 25 in which the active pharmaceutical composition is a pesticide.

27. A method as claimed in claim 26 in which the pesticide is a herbicide.

28. A method as claimed in claim 26 in which the pesticide is suitable for controlling or eradicating mammalian pests.

29. A method as claimed in claim 28 in which the pesticide is suitable for controlling or eradicating ectoparasites.

30. A method as claimed in claim 28 in which the pesticide is an acaricide.

31. A method as claimed in claim 30 in which the acaricide belongs to the amidine group of pesticides.

32. A method as claimed in claim 31 in which the pesticide is AMITRAZ.
